(19) United States
(12) Patent Application Publication
(10) Pub. No.: US 2004/0185115 A1
(43) Pub. Date: Sep. 23, 2004

(54) Emu oil based methods and compositions for skin ailments

(76) Inventors: Maurine Pearson, Pilot Point, TX (US); Teresa Leigh Barr, Port Townsend, WA (US)

Correspondence Address: Wendy K. Buskop, Buskop Law Group, P.C., Suite 500, 1717 St. James Place, Houston, TX, 77056, US

(21) Appl. No.: 10/439,889

(22) Filed: May 16, 2003

Related U.S. Application Data
(60) Provisional application No. 60/381,418, filed on May 17, 2002.

(51) Int. Cl. A61K 35/12

(52) U.S. Cl. 424/522; 514/389; 514/568

(57) ABSTRACT

Methods and compositions for treating skin ailments are described herein. Embodiments of the invention include a therapeutic composition. The thereapeutic composition is adapted for cleansing and treating skin and further includes an emu oil based composition. The emu oil based composition includes from about 20 wt. % to about 75 wt. % of refined sterilized emu oil, from about 0.01 wt. % to about 33 wt. % of one or more aromatic alcohols, from about 10 wt. % to about 25 wt. % of one or more benzoic acids, from about 0.2 wt. % to about 2.0 wt. % of allantoin, from about 0.025 wt. % to about 1.25 wt. % of methylparaben, from about 1.0 wt. % to about 13.0 wt. % of one or more alkyl esters and from about 0.01 wt. % to about 0.30 wt. % of propylparaben. Embodiments of the invention further include a composition including an aqeous emu oil based composition, wherein the aqueous emu oil based composition is adapted for cleansing and treating skin. Embodiments of the invention further include an absorbent article. The absorbent article includes a liquid permeable top sheet, wherein the liquid permeable top sheet is adapted to contact skin and is treated with an emu oil based composition. The absorbent article further includes a liquid permeable back sheet and an absorbent core disposed between the liquid permeable top sheet and the liquid permeable back sheet. Embodiments of the invention further include a method for preventing baby rashes and skin ailments. The method includes preparing an emu oil based composition and applying the emu oil based composition to an ailment selected from the group including baby rashes and skin ailments.

### CROSS-REFERENCE TO RELATED APPLICATIONS

This application claims priority to provisional U.S. Patent Application Serial No. 60/381,418, filed on May 17, 2002.

### BACKGROUND

1. Field of Invention

Embodiments of the invention relate to a composition adapted to treat skin ailments.

2. Description of Related Art

Found in the wilds of Australia, emus (Dromiceius novae-hollandiae) are the second largest members of the group of flightless birds. Emus can be raised like ordinary farm animals and used for their valuable products, which include very low fat meat, supple leather hides, decorative and nutritional eggs and rich oil.

Emu oil, a food by-product, is obtained from the fat of the emu and is an all-natural substance. As a result, emu oil contains high amounts of essential fatty acids, referred to herein as EFA's. EFA's produce energy in the process of oxidation and, as a result can govern growth, vitality and mental state of mind in humans.

Many references have been made to the efficaciousness of emu oil. U.S. Pat. No. 5,431,924, which is hereby incorporated by reference, describes using an emu oil extract diluted with hexane for treatment of the skin.

U.S. Pat. No. 6,193,987, which is hereby incorporated by reference, teaches a formulation for treating skin including a tincture of benzoin with emu oil. The tincture of benzoin is added in increments of 6-10 drops. The tincture of benzoin has a chemical composition of C₂H₅OH and an alcohol content of 79%, such that the benzoin is only 10.0 wt. % of the entire composition containing the emu oil.

U.S. Pat. No. 4,837,019, which is also incorporated by reference, teaches a composition that counteracts moisture loss and promotes healing of burned or sunburned skin and includes polyglyceryl methacrylate, glycerine, allantoin, panthenol, amino acid complex, and fibronectin in the composition. Allantoin is taught in the form of a complex of allantoin and N-acetyl dl methionine.

Specifically, this composition is taught to increase cellular respiration, improve a cell's water binding properties, and promote normal cell sloughing. The patented formula uses allantoin in the range from about 0.1% to about 0.5%, and preferably from about 0.1% to about 0.4% by weight.

Other patents also discuss uses of emu oil formulations, for example, U.S. Pat. No. 5,662,921 teaches the use of emu oil to prevent scarring when applied to a newly received cut or burn. U.S. Pat. No. 5,662,921 also covers a method on using emu oil to increase high-density lipoproteins to aid in the prevention of scarring of skin tissue and aid in treating scarred skin.

U.S. Pat. No. 5,958,384 teaches that topical or parenteral administration of emu oil to a mammal to stimulate the proliferation of skin, as well as rejuvenate photo-damaged skin. This formulation is also used to stimulate melanogenesis in the skin and treat disorders such as hypo-pigmentation.

Emu oil can be processed in a number of ways, including rendering, which means the oil is simply filtered, with the filtered oil possibly including contaminants. Further, filtered emu oil retains its natural yellow color and a slight odor. In addition, emu oil can be extracted and then diluted in other materials. Emu oil can also be sterilized to remove contaminants, color and odor, but is rarely performed due to degradation of the resulting oil.

Therefore, a need exists for sterile emu oil formulations that can be combined with other ingredients to form a creamy white and odor free composition. The resulting composition can be used in a variety of skin treatments, such as treating insect bites.

Further, there exists a need for a contaminant free emu oil formulation that can be used as a preparation and treatment for normalizing the skin cells for treatment of venomous snake bites, while remaining stable and usable over time without degradation.

### SUMMARY

Embodiments of the invention include a therapeutic composition. The thereapeutic composition is adapted for cleansing and treating skin and further includes an emu oil based composition. The emu oil based composition includes from about 20 wt. % to about 75 wt. % of refined sterilized emu oil, from about 0.01 wt. % to about 33 wt. % of one or more aromatic alcohols, from about 10 wt. % to about 25 wt. % of one or more benzoic acids, from about 0.2 wt. % to about 2.0 wt. % of allantoin, from about 0.025 wt. % to about 1.25 wt. % of methylparaben, from about 1.0 wt. % to about 13.0 wt. % of one or more alkyl esters and from about 0.01 wt. % to about 0.30 wt. % of propylparaben.

Embodiments of the invention further include a composition including an aqeous emu oil based composition, wherein the aqueous emu oil based composition is adapted for cleansing and treating skin.

Embodiments of the invention further include an absorbent article. The absorbent article includes a liquid permeable top sheet, wherein the liquid permeable top sheet is adapted to contact skin and is treated with an emu oil based composition. The absorbent article further includes a liquid permeable back sheet and an absorbent core disposed between the liquid permeable top sheet and the liquid permeable back sheet.

Embodiments of the invention further include a method for preventing baby rashes and skin ailments. The method includes preparing an emu oil based composition and applying the emu oil based composition to an ailment selected from the group including baby rashes and skin ailments.

### BRIEF DESCRIPTION OF DRAWINGS

FIG. 1 illustrates a flowchart of an embodiment of the invention.

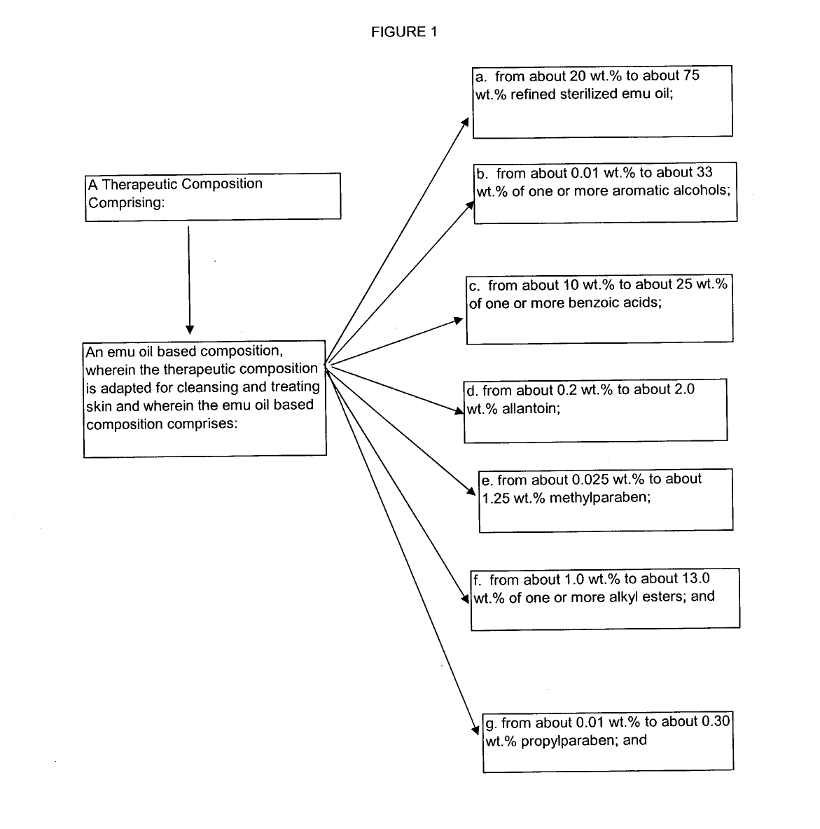

### DETAILED DESCRIPTION

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the pertinent art to make and use the inventions, when the information in this patent is combined with available information and technology. Various terms as used herein are defined below. To the extent a term used in a claim is not defined below, it should be given the broadest definition persons in the pertinent art have given that term as reflected in printed publications and issued patents.

A. Compositions

Embodiments of the invention include a composition for treating skin ailments. As used herein, the term “ailment” refers to any item on or in the skin that would benefit from treatment, such as but not limited to, skin conditions, e.g., diseases, burns, rashes and wounds. The composition generally includes a variety of components, but specifically includes from about 20 wt. % to about 75 wt. % of refined sterilized emu oil. Emu oil contains a variety of components, which aid in treating skin. For example, emu oil includes fatty acids, such as oleic acid, palmitic acid, elaidic acid, stearic acid, myristic acid, lauric acid, lignoceric acid, behenic acid and vaccenic acid. Two typical fatty acids are oleic and palmitic. An analysis of fatty acids in emu oil reveals that the oil contains approximately 70% unsaturated fatty acids. The composition and structure of the fatty acids of naturally occurring lipids generally have an even number of carbon atoms because they are synthesized from acetyl groups, each of which contains two carbon atoms. In most cases, the fatty acids are not found in free form, but instead are bound to other compounds to form fatty acid containing lipids, e.g., neutral lipids (triglycerides) sterols, phosphoglycerides such as lecithin, and sphingolipids such as sphingomyelin.

Although palmitic acid and stearic acid are the major saturated fatty acids found in animal and plant tissues, significant amounts of other saturated fatty acids such as myristic acid and lauric acid, occur in certain tissues, and lignoceric acid and behenic acid are found in high concentrations in brain sphingolipids. Small amounts of fatty acids with an odd number of carbon atoms are also known, e.g., margaric (17:0) and margaric oleic (17:1). A naturally occurring odd-carbon acid, identified as C₁₇, has the ability to cross the skin barrier and along with significant amounts of other saturated fatty acids. Amounts of fatty acids with an odd number of carbon atoms are also known, e.g., pentadecanoic acid and heptadeconoic acid. The present invention emu oil composition contains high amounts of octadecanoic acid, pentadecanoic acid and heptadeconoic acid. Naturally ocuirring heptadocenic acid is normally isolated in mutton and shark liver oil, and is novel and naturally occurring and as well present in high amounts within the emu oil.

The primary fatty acid found in emu oil is oleic acid, which is monosaturated and makes up over 40% of the total fatty acid contents. This fatty acid is a known enhancer for penetration and transdermal transportation of compounds through the pores of the epidermis, a membrane of the skin, and, thus, delivers the active ingredients into the lipid layer of the skin at the cellular level, faster than other known formulations.

The emu oil further includes essential fatty acids, such as myristic acid, palmitic acid, palmitoleic acid, margaric acid, margaric oleic acid, oleic acid, linoleic acid, linolenic acid, arachidic acid, elaidic and vaccenic and eicosinoac acid. Emu oil further contains both of the two essential fatty acids (EFA's) that are important to human health, linolenic and alpha-linolenic acid. As used herein, “essential fatty acids” are those fatty acids which humans must obtain from their diet because the human body cannot manufacture them, hence, making them essential as transdermal supplements to nourish and proliferate the normalization of skin cells.

Stearic acid is also called octadecanoic acid, one of the most common long chain fatty acids, found in combined form in natural animal and vegetable fats. Commercial stearic acid is a mixture of approximately equal amounts of stearic and palmitic acids and small amounts of oleic acid. In nature, stearic acid occurs primarily as a mixed triglyceride, or fat, with other long-chain acids and as an ester of fatty alcohol. It is much more abundant in animal fat than in vegetable fat, for example, lard and tallow often contain up to 30% stearic acid.

The fats found in land animals have a higher percentage of side chains than do the fats in sea animals. Although unsaturated fats are less efficient storage sites for food energy because they have fewer CH bonds than do saturated fats, they have a distinct advantage for animals that live in cold water. Saturated fats melt at higher temperatures than unsaturated fats. In cold waters, sea animals with solid fats would have the reduced ability to move. This theory may prove it easier to transport unsaturated fats through the skin structure and membrane into the lipid layer, rather than a saturated fat.

Emu oil further includes iodine, which demonstrates both antiseptic and germicide properties. An analysis of the emu oil reveals the iodine content to be about 72.8%. Typically, the therapeutic index for iodine is among the highest of the antiseptics. Unfortunately, iodine burns are common and largely the result of the conventional tinctures and solutions having iodine concentrations that are higher than tolerated by certain skin types. However, the fatty acids present in emu oil function as a buffer and neutralize the skin against the harmful side effects of iodine in humans and animals, while taking advantage of the germicide effect.

Preferably, the composition includes from about 40 wt. % to about 70 wt. % of the refined sterilized emu oil. More preferably, the composition includes from about 60 wt. % to about 70 wt. % of the refined sterilized emu oil.

The composition additionally includes from about 10 wt. % to about 33 wt. % of one or more aromatic alcohols, which inhibit bacterial growth. Preferably, the composition includes from about 15 wt. % to about 30 wt. % of the one or more aromatic alcohols, and more preferably, the composition includes from about 15 wt. % to about 20 wt. % of the one or more aromatic alcohols. The one or more aromatic alcohols can include any aromatic alcohol, such as benzyl alcohol, propyl alcohol and methyl alcohol. Preferably, the one or more aromatic alcohols include benzyl alcohol. More preferably, the one or more aromatic alcohols include sterilized benzyl alcohol.

Benzyl alcohol is listed in a summary of ingredient categories and testing as a category 1 analgesic, anesthetic, and anti-puritic active ingredient. One example of usable benzyl alcohol is Benzyl Alcohol NF-Benzenemethanol; Phenylcarbinol CH₂-OH Benzyl Alcohol C₇H₈. The benzyl alcohol, which can be used within the scope of the invention, involves using esters of benzoic and cinnamic acids in storax, Peruvian balsam, and tolu balsam. A product currently on the market can be used which is made synthetically from benzyl chloride by distilling it from an aqueous solution of potassium carbonate with thorough agitation.

The composition can further include from about 10 wt. % to about 33 wt. % of a one or more benzoic acids. Preferably, the one or more benzoic acids include benzoin. Benzyl alcohol and benzoic acid possess beneficial bacteria retarding properties, but typically are irritating to the skin and therefore are not generally included in conventional skin treatment products, or are present in only small amounts, e.g., less than about 10 wt. %. The compositions described herein include benzyl alcohol and benzoic acid in an amount of from about 15 wt. % to about 33 wt. %. Although the alcohol content of the emu oil based composition is higher than typical compositions used to treat skin conditions, the emu oil used in the embodiments described herein, along with the allantoin, are synergists and generally buffer the effect of the high alcohol content.

Specific benzoic acids having between 12 and 15 carbon atoms, and alkyl esters can be added to an embodiment for the formula. For example, flowers of Benzoin; flowers of Benjamin; Phenylformic Acid, and Benzoic Acid, which is C₇ H₆ O₂, can be used. Benzoic Acid is the simplest acid of the aromatic series. Although the acid is of minor significance as a medicinal agent, it derivatives and salts constitute an important group of valuable medical agents. The addition of this component to the formula, enable the invention to act as an antifungal agent chiefly in combination with salicylic acid as well as being an anesthetic. When the emu oil contains enough benzoic acid it can then be used in the treatment of athletes' feet and to a lesser extent in the management of ringworm, for humans and animals. Benzoin is preferably used in amounts between 10-33 wt % and most preferably 10 wt %.

Various antimicrobial drugs can be added to the Emu oil, including but not limited to: methylparaban or a benzoic acid, or an alkyl ester such as 4-hydroxy-, methyl ester; or possibly Solbrol made by Charkit Chemical Corporation, P.O. Box 1725, Darien, Conn. 07407; Methyl Parasept made by Charkit Chemical Corporation, P.O.

Box 1725, Darien, Conn. 07407; Nipagin or even a Methyl p-hydroxybenzoate (99-76-3) C8 H8 O3. An antimicrobial additive can be formed by esterifying para-hydroxybenzoic acid with methanol using known techniques. The para-hydroxybenzioc acid is obtained by passing carbon dioxide under pressure into dry potassium phenolate heated to about 200 degrees. The resulting potassium salt is decomposed with HCl yielding the free parabic acid. These components can be added in amounts ranging from 0.25-1.25 wt % and most preferably, 2.5 wt %.

The composition further includes from about 0.2 wt. % to about 2.0 wt. % of allantoin, from about 0.025 wt. % to about 1.25 wt. % of methylparaben, from about 1.0 wt. % to about 13.0 wt. % of one or more alkyl esters and from about 0.01 wt. % to about 0.30 wt. % of propylparaben.

Still another ingredient, Allantoin can be used, specifically, Allantoin-5-Ureidohydantoin C₄ H₆ N₄ O₃ can be added to the formula. Allantoin is used topically as a vulnerary to stimulate tissue repair in suppurating wounds, resistant ulcers, acne seborrhea, and basic dermatological infections. It is also included in some topical preparations for oral and dental use. It is frequently combined with antiseptics and antifungal drugs. The silver salt is used in the topical treatment of extensive burns. Typically, 0.2 to 2.0% of this ingredient can be used in the formula, particularly when the invention is used as creams, lotions or shampoo.

Methylparabens and other related esters are of para-hydroxybenzoic acid which are odorless and harmless to the skin can be employed in the formula. A combination of two or more esters of para-hydroxybenzoic acid has a “synergistic” antiseptic value, i.e. the antiseptic effect of the combination is greater than the total effect as calculated from the values of the individual components; thus a preparation containing 0.15% of the propyl ester (propylparaben) and 0.05% of the benzyl ester has a stronger antiseptic value than 0.2% of either ester alone. The benzyl ester has a high antiseptic value and is suitable for the preparation of antiseptic creams. The preferred amount of alkyl ester for use in the invention is between 1-13 wt % and most preferably 3 wt %.

Preferably, the composition includes from about 1.5 wt. % to about 2.0 wt. % of allantoin, from about 1 wt. % to about 1.25 wt. % methylparaben, and/or from about 0.25 wt. % to about 0.3 wt. % propylparaben.

In one embodiment, the composition includes from about 2.5 wt. % to about 3 wt. % of the one or more alkyl esters. In another embodiment, at least one of the one or more alkyl esters are branched.

Parahydroxybenzoic acid esters and mixtures of methylparaben and propylparaben can be used in the invention with excellent and unexpected results. They are commonly used as antimicrobial preservatives can the amount of their use is contemplated to be in ranges of methylparaben 0.025-0.2 wt percent, with a preferred range of 0.1 to 01.25% and propylparaben in the range of 0.01 to 0.4 wt %, most preferably, approximately 0.3%-0.04 wt %.

The composition can further include an acid, such as lineolic acid, to aid in normalizing skin tissue. Supplementing the emu oil based composition with linoleic acid aids in fortifying the skin to slowly to heal wounds, strengthen and rebuild the skin.

Preferably, the composition includes from about 20 wt. % to about 40 wt. % of the acid, and more preferably from about 28 wt. % to about 32 wt. % of the acid.

The composition can additionally contain antimicrobial agents, which operate to control wound infection, as a topical anti-infective, and eliminate microbial growth and necrotic tissue, all of which interfere with tissue repair. In addition, the formula can include a topical analgesic/anesthetic at active levels (as set by FDA monograph, Section 348 of 21 CFR), which operate as a topical pain control product.

Additional preservatives can be added to the inventive formula such as Imidazolidinyl Urea in concentrations ranging from 0.05 to 1.0%.

One embodiment of the invention includes a composition for treating skin ailments, wherein the composition is adapted for spray application to the skin ailment. The composition includes from about 60 wt. % to about 70 wt. % of refined sterilized emu oil, from about 10 wt. % to about 20 wt. % of benzyl alcohol, from about 10 wt. % to about 25 wt. % of benzoin, from about 1 wt. % to about 2 wt. % of allantoin; from about 0.75 wt. % to about 1.25 wt. % of methylparaben, from about 1 wt. % to about 5 wt. % of one or more alkyl esters and from about 0.04 wt. % to about 0.30 wt. % of propylparaben.

Embodiments of the present invention also relate to use of the compositions described herein as a local anesthetic by injection and by application to mucous membranes. Externally the composition can be applied as an ointment or as a lotion in topical preparations and used as a bacteriastatic agent in various parenteral preparations. Externally the formula can also be applied to nasal passages and gum tissues of humans and animals.

The compositions described herein can be packaged in a variety of forms, such as a cream or spray. In addition, the compositions can be packaged in a roll-on form. Further, the compositions can be packaged in wipe on form. When in wipe on form, the wipe can be impregnated with the composition and packaged in an appropriate container or towelette to prevent the wipe from drying out.

The compositions can further be packaged as a drenching formula, similar to that of the preferred spray formula. When in drenching form, the application site is drenched with the composition and packaged in an appropriate container to squeeze onto afflicted area.

It has been very difficult to create spray on emu oil, as it coagulates and gums up the spray. In addition, for the treatment of fresh bites, it has been hurtful to use a spray with an alcohol, as the alcohol can cause pain on the wound or the burn until the alcohol evaporates. Accordingly, particular embodiments of the present invention are a combination of two elements, particularly a spray and that of an alcohol which can “burn” the skin with an oil that usually can not be “sprayed” because it is too thick, and yet the present formulation has been created which has these two features and can be used for a variety of skin treatments, when application by touch would hurt the person.

Preferably, the compositions are in a spray form for easy application to the skin.

In addition, the emu oil is refined to a color of white, and can further be refined to a contaminant level of about 3% or less.

The compositions described herein uniquely can be sterilized. Traditionally, sterilization has broken down the components of oils, which contain these types of fatty acids. The objective of a sterilization process is to remove or destroy all microorganisms in or on a preparation and to assure in this way the preparation is free of infectious hazards when used with a patient. Since the variety and amounts of the variety and amounts of sterile materials required for health care have increased in significant proportions, sterilization technology has become increasingly important. Alternatively, if sterilization of the oil is not preferred, then a disinfectant can be added to the formula to render the skin noninfectious. A usable disinfectant may be an antiseptic or a germicide.

The guidance on validation of the manufacture of sterile products can be found in the FDA's submission Documentation for Sterilization Process Validation for Human and Veterinary Drug Products (November 1994), which are hereby incorporated by reference.

The emu oil based compositions described herein perform a number of functions when applied to skin. For example, the composition includes beneficial ingredients capable of creating healthier cells, unexpectedly normalizing and stabilizing the skin at an accelerated rate. The cause of the unexpected increase is unknown, but it is believed that emu oil is biologically active to human skin, as demonstrated in Table 1 below. In addition, the methods and compositions described herein inhibit bacterial growth in the affected area, such as in the snake bite, thereby preventing the spread of bacteria and flaking. As a result, the smooth appearance of the skin can be restored. In addition, the composition stabilizes the erythma and exfoliation of the skin, therefore helps to control itching and pain as well as infection of the skin. Further, the compositions described herein increase the pliability of the affected area and thus improve pigmentation and vascularity of the skin.

TABLE 1

| Fatty Acid | Carbon Ratio | Emu Oil | Human Skin Oil |
|---|---|---|---|
| Myristic | C:14:0 | 0.3% | 2.1% |
| Palmitic | C:16:0 | 20.3% | 20.2% |
| Palmitoleic | C:16:1 | 3.2% | 3.8% |
| Margaric | C:17:0 | 0.2% | |
| Margaric oleic | C:17:1 | 0.1% | |
| Stearic | C:18:0 | 10.1% | 11.2% |
| Oleic | C:18:1 | 51.6% | 30.8% |
| Linoleic | C:18:2 | 13.1% | 15.1% |
| Linolenic | C:18:3 | 0.5% | 0.3% |
| Arachidic | C:20:0 | 0.1% | |
| Eicosinoac | C:20:1 | 0.5% | |

Although inflammation is the normal body response to healing chronic ulcers and burn wounds, which causes scar tissue to form, the compositions described herein provide moisture in areas where sebaceous glands are depleted or currently dysfunctional. This increases the pliability of the wound area and, thus improves pigmentation and vascularity. In addition, the emu oil based composition, when topically applied, is believed to increase the synthesis of DNA in the epidermis, demonstrating an increase in the proliferative activity of the dermis.

Further, the composition is stable when exposed to air. It has been observed that sterile emu oil, as described herein, can be used with long lasting effects, e.g., stored for several months, and without the degradation experienced previously.

B. Treatment of Insect Penetrations

One embodiment of the methods and compositions described herein is adapted for treating a variety of insect penetrations in skin. The composition includes any of those compositions described above, but which is adapted for topical application to an area of the skin affected by the insect penetration.

The insect penetration can include a variety of insect penetrations, such as bee stings, tick bites, ant bites, such as fire ant bites, and mosquito bites. In addition, the composition can treat spider bites, such as those from the brown recluse spider and the black widow spider.

The brown recluse spider is found mainly in the southern and Midwestern states especially Arkansas, Oklahoma and Missouri. To date, infested Ohio counties have had a history where this spider probably shipped into the home in furniture, appliances, storage cartons, boxes, old clothes and other household goods. The brown recluse spider is not aggressive. Most bitten people have directly contacted the spider when putting on clothing or shoes not used for long periods of time. They usually occur in houses in undisturbed areas. People have been bitten after sleeping in an unused bed after rolling over onto the spider or others after accidentally touching the spider when cleaning out unused storage areas. Fatalities are rare, but bites are most dangerous to children, elderly and those in poor physical condition. The formulation can be used to treat these spider bites. Both the male and female brown recluse spiders are similar in appearance and equally toxic.

Spiders can survive long periods of time without food or water and may live as long as two years. This spider is most active at night when it comes out in search of food consisting of cockroaches and other small insects. During the day, time is spent in quiet, undisturbed places such as bathrooms, bedrooms, closets, basements and cellars. The spiders sometimes take shelter under furniture, appliances and carpets, behind baseboards and door facings, or in corners and crevices. Some have been found in stored clothing, old shoes, on the undersides of tables and chairs, and in folded bedding and undisturbed towels stored for long periods of time. Outdoors, the spider may be found in sheltered corners among debris, in wood piles, under loose bark and stones, in old barns, storage sheds and garages. These spiders are very adaptable and may be active in temperatures ranging from 45 to 110° F.

The severity of a person's reaction to the bite depends on the amount of venom injected and individual sensitivity to it. Bite effects may be nothing at all, immediate or delayed. Some may not be aware of the bite for 2 to 8 hours, whereas others feel a stinging sensation usually followed by intense pain if there is a severe reaction. A small white blister usually rises at the bite site surrounded by a large congested and swollen area. Within 24 to 36 hours, a systemic reaction may occur with the victim characterized by restlessness, fever, chills, nausea, weakness and joint pain. The affected area enlarges, becomes inflamed and the tissue is hard to the touch. The spider's venom contains an enzyme that destroys cell membranes in the wound area with affected tissue gradually sloughing away, exposing underlying tissues. Within 24 hours, the bite site can erupt into a “volcano lesion” (a hole in the flesh due to damaged, gangrenous tissue). The open wound may range from the size of an adult's thumbnail to the span of a hand. The sunken, ulcerating sore may heal slowly up to 6 to 8 weeks. Full recovery may take several months and scarring may remain. Plastic surgery and skin grafts are sometimes required. The inventive method uses the formulation to treat bites from this type of spider.

Usually the only treatment for this kind of spider bite is a high dose of cortisone-type hormones to combat hemolysis and other systemic complications. Brown recluse spider bites can cause significant cutaneous injury with tissue loss and necrosis. Less frequently, more severe reactions develop, including systemic hemolysis, coagulopathy, renal failure, and, rarely, death. Brown recluse venom, like many of the other brown spider venoms, is cytotoxic and hemolytic. It contains at least 8 components, including enzymes such as hyaluronidase, deoxyribonuclease, ribonuclease, alkaline phosphatase, and lipase. Sphingomyelinase D is thought to be the protein component responsible for most of the tissue destruction and hemolysis caused by brown recluse spider envenomation. The intense inflammatory response mediated by arachidonic acid, prostaglandins, and chemotactic infiltration of neutrophils is amplified further by an intrinsic vascular cascade involving the mediator C-reactive protein and complement activation. These and other factors contribute to the local and systemic reactions of necrotic arachnidism. Although numerous cases of cutaneous and viscerocutaneous reactions have been attributed to spiders of the genus Loxosceles, confirming the identity of the envenomating arachnid is difficult and rarely accomplished. Either a few minutes or a few hours after a bite, the skin around the area will become red and swollen, but the degree of reaction can range anywhere from negligible to severe. In all but the mildest reactions, the bite of a brown reclusive causes significant pain. The wound may take months to heal, and much of the tissue around the bite dies, leaving a depressed scar about an inch across. Plastic surgery may be required to restore the area to a normal appearance. Although the bite is rarely fatal, deaths do occasionally occur. Usually these are either the result of an allergic reaction or the consequence of secondary infections, because the venom of the brown recluse compromises the normal functioning of the immune system. The spray on formulation can assist with the treatment of these bites and associated symptoms to reduce itching and promote cell regeneration while killing harmful bacteria spores that try to spread in connection with this ailment.

The Black Widow Spider, common name for any of several related long-legged, smooth-bodied spiders, chiefly inhabiting the Tropics, is also common in the southern United States and found as far north as Canada and has a bite that can be treated with this formulation to stop itching and lesions. The Black Widow is about 1.2 cm (about 0.5 in) long and is jet black, with an hourglass-shaped red mark on the underside of the abdomen. Males are only about half as long and usually have four pairs of red dots along the sides of the abdomen. The female's bite, poisonous to humans, is followed by local pain and swelling, nausea, and difficulty in breathing and is sometimes fatal. The venom, a neurotoxin, generally affects children more severely than adults. The spider, however, is not aggressive and bites humans only defensively.

At least three other species found in the United States are also poisonous to humans:

the brown, or gray, widow spider; the red widow spider, with irregular red and yellow markings; and the northern widow spider, with a row of red spots above and two red bars on the underside of the abdomen. Scientific classification: Black widow spiders make up the genus Latrodectus in the family Theridiidae. The name is applied especially to the familiar North American species Latrodectus mactans. The brown, or gray, widow spider is classified as Latrodectus geometricus, the red widow spider as Latrodectus bishopi, and the northern widow spider as Latrodectus various. According to Willia Gertsch, curator of spiders at the American Museum of Natural History, New York, the venom of the black widow spider is 15 times as toxic as the venom of the prairie rattlesnake. However, only a minute amount of the toxin is injected with a single bite by the spider, while the relatively large amount of injected rattlesnake venom results in about 15 to 25 percent mortality among those bitten.

The severity of a person's reaction to the bite depends on the area of the body bitten, amount of venom injected, depth of bite, seasonal changes and temperature. The bite feels like a pin prick or is not even felt. At first, there may be only slight local swelling and two faint red spots surrounded by local redness at the bite. Pain becomes intense in one to three hours and may continue up to 48 hours. Pain usually progresses from the bitten member up or down the arm or leg, finally localizing in the abdomen and back. The abdominal muscles may become rigid and board-like with severe cramps which resemble appendicitis. There may be pain in the muscles and soles of the feet, and eyelids may become swollen. Other symptoms may be nausea, profuse perspiration, tremors, labored breathing and speech, and vomiting. During this time, a feeble pulse, cold clammy skin, unconsciousness, convulsions and even death may result if the victim does not receive medical attention immediately. Additional complications may occur due to the infection of the bite. However, with some untreated individuals, symptoms may diminish in several hours and be gone in several days after agony. Bites are uncommon and serious long-term complications or deaths are rare. Only four deaths were officially attributed to black widow bites in the United States from 1960-69. A physician can give specific antivenom or calcium gluconate to relieve pain. Healthy people recover rapidly in two to five days. Although the acute systems are not likely to be treatable with the formulation, the skin conditions are treatable with the novel formulation.

The composition can further be used to treat the bites of ticks. Ticks are actually a specialized group of mites and share many features with other mites. In general, they are larger than most mites, ranging from about 0.2 to 0.6 cm (about 0.08 to 0.24 in) in length, although females may be 1 cm (0.4 in) or more in diameter when fully engorged with blood. The adult tick has a mitelike body with a tough skin and four pairs of clawed legs; tick larvae have only three pairs of legs. The mouthparts consist of a paired anchoring organ, or rostrum, covered with backward-curving hooks and equivalent to the pedipalps of other arachnids; and a pair of sharp mandibles that move back and forth in two longitudinal channels on the rostrum. Ticks spend a great deal of time waiting for their hosts. They are particularly sensitive to carbon dioxide and movement—signals that a host is nearby. Their grasping forelegs allow them to climb on a host. They quickly find a protected spot on the host's body, sink their mouthparts into the flesh, and begin to feed. When full, they drop off the host. In some species, adult males and some nymph stages do not feed. Ticks are divided into two families: hard ticks and soft ticks. In hard ticks, the mouthparts are visible from above. Hard ticks are parasites primarily of mammals but are also found on birds and reptiles. The nymphs may feed on a different host species in each developmental stage; in each stage, the nymph feeds only once. The adult female lays a single large batch of eggs after her final meal. The American dog tick is perhaps the most familiar North American hard tick. Another important species is the deer tick, which is known to transmit Lyme disease to humans. The invention can treat the conditions cause by Lyme disease.

The Australian Paralysis Tick, Ixodes holocyclus, is an important life-threatening parasite of man and animals. It is also the tick most commonly found on dogs, cats and humans on the East Coast. The tick's paralyzing toxin has been estimated by Stone (1997) to affect as much as 100,000 domestic animals annually, with up to 10,000 companion animals being referred to veterinary surgeons for treatment. In human population health terms, tick envenomation is a far greater medical problem for children than snake or spider bite (Pitt, 1999). Humans also face a small but significant risk of acquiring tick-transmitted infectious diseases.

Fire ants are originally from South America, where dozens of fire ant species exist. There have been two introductions of fire ants which have led to current problems. The first was the black imported fire ant, Solenopsis richteri, which w as likely brought from Argentina in ship ballast to Mobile, Ala., in 1918. A second and by far the most damaging species, Solenopsis invicta, known as the red imported fire ant, arrived in Mobile sometime in the late 1920s or early 1930s, probably also in ship ballast.

Additionally, several fire ant species were present in the U.S. prior to the importation of the black and red imported fire ants. These native species were sometimes a problem, and still can be a problem in areas not yet invaded by the imported form s. Secondly, the presence of many species of fire ants in both South and Central America and many islands means that they could reinvade the United States. Some of these species are considered potentially a bigger problem than these already present. For the most part, fire ants often occupy the same areas where we work, live and play. It is estimated that about 40 million people are in potential conflict with fire ants, almost on a daily basis. Fire ants are so named because their venom induces a painful, fiery sensation. When disturbed, fire ants are very aggressive. The ant grips the skin with its mandibles or jaws and stings its victim several times in a circular pattern around the point of mandible attachment. Because of the ant's aggressive nature and capacity for multiple stings, an attack usually results in several stings. Some people who are stung experience only local reaction and temporary discomfort but, in most, a swollen red area will occur followed by a sterile pustule within 24 hours. Although the venom is bactericidal, secondary infections due to scratching may occur.

While there are more than 13 genera of mosquitoes in the United States, most pest mosquitoes belong to one of three: Aedes, Culex, or Anopheles. Some Aedes mosquitoes are called “floodwater mosquitoes” because they lay their eggs singly on damp soil or vegetation in areas that are periodically wet. Other Aedes species prefer to lay their eggs in artificial containers or tree holes. Again the eggs are laid just above the water line and hatch once they are inundated.

The Asian tiger mosquito, Aedes albopictus, first appeared in the United States in 1985. Its rapid spread is of concern because it is known as a disease-carrying mosquito in its native Asia. It also breeds readily in water-filled containers, so breeding sites are commonly available. These mosquitoes breed in quiet standing water of all types, ranging from containers to larger pools.

Culex species prefer polluted standing water with a large amount of organic material. Eggs are laid on the surface of the water in “rafts,” usually of 100 or more eggs. While Aedes and Anopheles mosquitoes have a pointed tip at the end of the abdomen, Culex mosquitoes have a blunt tip. Anopheles mosquitoes can be distinguished from Aedes and Culex mosquitoes in several ways: (1) Anopheles have patterned wings, (2) adult Anopheles females have palps that are almost as long as their proboscis, (3) adults rest on surfaces with their head lower than the abdomen while Aedes and Culex species rest with the head and abdomen parallel to the surface, and (4) the Anopheles larvae float parallel to the water surface rather than hanging down at an angle.

Several flies bite people and can be annoying pests. These include black flies, biting midges and deer flies. Bites can be painful or produce swelling and intense itching as the result of injected saliva. Biting flies attack people, cattle, horses and pigs tend to be concentrated around the ears and head. In addition to the blood loss, effects of the insect saliva can cause a variety of problems, with swelling and intense skin irritation most common.

Biting midges are minute insects, often called “no-see-ums” because they are so small, usually less than ⅛ inch. Despite their size, they can be painful biters and highly annoying. Two common biting midges that cause problems in Colorado are Leptoconops species and Culicoides species.

Deer flies Chrysops spp, Silvius are moderate-sized insects. Most common species are gray or light brown, sometimes with patterned bodies and wings, and have large colored eyes. Deer flies are biters, produce a painful bite, and frequently draw blood in the process.

Snipe flies Symphoromyia are close relatives of deer flies. They are found near forested areas of higher elevations of the state. Snipe flies can be vicious biters and bite mostly during the day.

The stable fly Stomoxys calcitrans is a blood-feeding pest known to attack almost any kind of warm-blooded animal. It is a major pest of confined livestock throughout the world. It looks like the common house fly except that its mouthparts are adapted for biting and sucking blood. The stable fly feeds by inserting its proboscis or beak through the skin and then sucking blood from its host.

The horn fly Haemotobia irritans is about half the size of the stable fly, and is a more injurious pest of cattle. It spends most of the day on its host, biting and feeding on blood 20 to 30 times during a 24 hour period. These flies can occur in groups of several hundred or more on an individual host and cause severe irritation and economic loss. The horn fly is not considered to be a pest of people, although it will sometimes bite people who are close to infested animals.

Another embodiment includes a method of treating an insect penetration. The method generally includes applying the emu oil based composition described above to an area of skin affected by the insect penetration.

The composition aids in quickly reducing pain and swelling associated with insect penetrations in the skin.

C. Treatment of Skin Conditions

Embodiments of the invention can further be used to aid in treating skin ailments, such as psoriasis, pruritis, acne, dermatitis, dandruff, anthrax and herpes, as well as guarding against contact bacteria such as streptococci, impetigo and smallpox. The embodiments can further be used to cleanse and treat the skin ailments of children, such as diaper rash, chicken pox and scarlet fever. When the composition is adapted for use on children, the aromatic alcohol content can be reduced to less than 15 wt. %, preferably less than 10 wt. %, or less than 7 wt. % or less than 5 wt. %, or more preferably less than 3 wt. %.

For example, embodiments of the invention include a therapeutic composition adapted for cleansing and treating skin. The therapeutic composition includes the compositions described herein. The therapeutic composition is preferably adapted to inhibit bacterial growth on skin.

The composition can further include an aqeous emu oil based composition adapted for cleansing and treating skin

Embodiments of the invention further include a method for preventing diaper rash. The method includes preparing an emu oil based composition, as described herein, and applying the emu oil based composition to the diaper rash.

Psoriasis is a chronic skin disease that generally appears as patches of raised red skin covered by a flaky white buildup. Although the exact cause is unknown, Psoriasis is believed to be related to faulty signals sent by the body's immune system. These signals accelerate the growth cycle in skin cells, which pile up on the surface when the body can't shed them fast enough. Psoriasis is not contagious no one can transmit it from another person. It has a genetic component that makes certain people more likely to develop it, but often an external or environmental trigger is necessary to make psoriasis appear. These triggers may include emotional stress, injury to the skin, some types of infection and reaction to certain drugs. The most common form of psoriasis is called plaque psoriasis. About 80 percent of people with psoriasis have this type. Plaque psoriasis can appear on any skin surface, although the knees, elbows, scalp, trunk and nails are the most common locations. There are several other types of psoriasis, and between 10 percent and 30 percent of people with psoriasis also develop a related form of arthritis, called psoriatic arthritis. It is an object of this invention to normalize and stabilize the skin cell growth and to prevent irritation and scratching to infection. The present invention can be used to treat psoriasis either as a spray on formulation or non-spray formulation.

Plaque Psoriasis is the most typical form of the disease and four out of five people with psoriasis have this type. A plaque is the name used to describe the well-defined patches of red, raised skin, and the word lesion is also commonly used. The technical name for plaque psoriasis is psoriasis vulgaris (vulgaris means common). The flaky, silvery white buildup on top of the plaques is called scale; it is composed of dead skin cells. This scale comes loose and sheds constantly from the plaques. Skin affected with psoriasis is generally very dry, and other possible symptoms include skin pain, itching and cracking. The normal composition of the emu oil with the formulation described can be used to treat specifically plaque psoriasis.

The compositions described herein can also be used to treat other ailments. In particular, the composition can be used to treat guttate psoriasis, which resembles small, red, individual drops on the skin (the word guttate comes from the Latin word meaning “drop”). These lesions generally appear on the trunk and limbs, and sometimes on the scalp, and they are usually not as thick or as scale-covered as plaque psoriasis. Guttate psoriasis often starts in childhood or young adulthood, and it may be triggered by an infection of some sort. Guttate psoriasis often comes on quite suddenly. It is sometimes preceded by a streptococcal infection of the throat, also known as strep throat. However, a variety of conditions have been known to bring on an attack of guttate psoriasis. In a study of 100 pediatric psoriasis patients, it was found that outbreaks of guttate psoriasis had been preceded by sore throats or tonsillitis, colds, chicken pox, physical traumas, psychological stress, illnesses and administration of drugs to prevent malaria. This form of psoriasis may resolve on its own, leaving a person free of further outbreaks, or it may clear for a time only to reappear later as patches of plaque psoriasis. Sometimes guttate flares in children continue throughout childhood, and the flares are often linked to repeated bouts of strep infection or other upper respiratory illnesses. Some physicians prescribe antibiotics to help prevent an occurrence of an infection that can cause the outbreak of guttate psoriasis. A course of antibiotics is often tried for initial outbreaks of guttate psoriasis. Persistent cases can be treated with moisturizers or stronger topical agents (coal tar, corticosteroids, topical vitamin D3 derivatives or topical retinoids). Bland ointments are considered the safest treatment during the acute eruptive stage of guttate psoriasis. However, people with guttate psoriasis may find it tedious to apply topical preparations to the many small spots on their skin. Ultraviolet light treatment with UVB or PUVA is usually effective for guttate psoriasis, particularly in combination with topical agents. Only in severe cases will physicians prescribe systemic medications for this type of psoriasis, although sometimes a short course of one of these agents may result in rapid and prolonged clearing.

The compositions described herein can also be used to treat inverse psoriasis, which is also called flexural psoriasis, and is often found in the armpits, groin, under the breasts and in other skin folds around the genitals and buttocks. This type of the disease appears as smooth, dry areas of skin that are red and inflamed but do not have the scaling associated with plaque psoriasis. Inverse psoriasis is particularly subject to irritation from rubbing and sweating because of its location in skin folds and tender areas. It is more common and troublesome in overweight people. Treatment can be difficult due to the sensitivity of skin in these fold areas. Steroid creams and ointments are considered very effective, but they should not be occluded (covered) with plastic dressings. Overuse or misuse of steroids, particularly in skin folds, can result in side effects, including thinning of the skin and stretch marks. Because these areas are prone to fungal infections, physicians sometimes use diluted topical steroids in combination with other medications, such as 1% or 2% hydrocortisone with anti-yeast or anti-fungal agents. Other topical agents, such as vitamin D3 derivatives, retinoids, coal tar or anthralin, can be somewhat effective in treating psoriasis in skin folds, but they may also be irritating. They should be used with caution and under the direction of a physician. Finally, if flexural psoriasis is worsened by surface growth of yeast, a trial of oral fluconazole may be of some value. People with severe, incapacitating flexural psoriasis may require systemic drugs such as methotrexate to control the condition.

The compositions described herein do not contain steroids and do not need to be occluded, have anti bacterial qualities that do not irritate the skin, and still control the surface growth of yeast.

Still another psoriasis which can be treated with the composition is erythrodermic psoriasis. This psoriasis is a particularly inflammatory form of psoriasis that often affects most of the body surface. It is the least common form of the disease. It most commonly appears on people who have unstable plaque psoriasis, where lesions are not clearly defined. The erythrodermic form of psoriasis is characterized by periodic, widespread, fiery redness of the skin. The erythma (reddening) and exfoliation (shedding) of the skin are often accompanied by severe itching and pain. Swelling may also develop. Erythrodermic psoriasis throws off the body chemistry, causing substantial protein and fluid loss that can lead to severe illness. Edema (swelling from fluid retention) may also develop along with infection. The body's temperature regulation is often disrupted. Infection, pneumonia and congestive heart failure brought on by erythrodermic psoriasis can cause death. People with this condition are often hospitalized. This form of psoriasis can occur abruptly as the initial sign of psoriasis, or come on more gradually in people with plaque psoriasis. The reason psoriasis becomes more generalized is not understood, although there are some known triggers. These include abrupt withdrawal of steroids (usually systemic); an allergic, drug-induced rash that brings on the Koebner response (a tendency for psoriasis to appear on the site of skin injuries); and severe sun burning. Initial therapy usually includes medium-potency topical steroids and liberal moisturizers, combined with oatmeal baths and bed rest. Careful attention is paid to restoring and maintaining fluids in the body. This is often successful in bringing the condition under control within a few days. Methotrexate, acitretin (Soriatane) or cyclosporine can be used as a means of bringing severe cases under control. Use of systemic steroids for erythrodermic psoriasis is controversial, and if used, they should be tapered off slowly, because stopping them suddenly can trigger a worsening of psoriasis. For acute cases, systemic steroids combined with methotrexate can be useful if a person's condition is monitored carefully during the necessary steroid tapering-off period. Combination therapies are often used because the medications needed to address an erythrodermic crisis have potentially serious side effects. Sometimes, antibiotics are added to combat infections. UVB (sometimes used in combination with coal tar) or PUVA therapies are used only after the initial inflammation has subsided. Once the erythrodermic flare passes, a person's psoriasis usually reverts to how it looked before the flare.

The composition can be used to treat pustular psoriasis which can spread over wide areas of the body and is also known as von Zumbusch pustular psoriasis. In this relatively rare form of the disease, widespread areas of reddened skin (erythema) develop, and the skin becomes acutely painful and tender. Pustules—blisters of non-infectious pus—may appear on the skin, dry, then reappear in repeated cycles lasting several days. The onset of von Zumbusch pustular psoriasis can be abrupt. Within as little as a few hours after the skin becomes tender, the pustules appear. The pustules then dry and peel over the next 24 to 48 hours, leaving the skin with a glazed, smooth appearance. A fresh crop of pustules may then reappear. Eruptions often come in repeated waves that last days or weeks. The pus inside psoriasis pustules consists of white blood cells. It is not an infection and it is not contagious. The method of using the invention to treat skin for this disease is particularly effective in that it stabilizes and normalizes blisters and keeps them from reappearing or becoming infectious.

Von Zumbusch psoriasis can be triggered by an infection, sudden withdrawal of steroids (usually systemic), pregnancy (this form is sometimes called “impetigo herpetiformis”) and drugs such as lithium, propanlol (Inderal) and other beta-blockers, iodides or indomethacin (Indocin). A person can have a history of plaque psoriasis for years and then develop recurrent episodes of von Zumbusch. This form can cause fever, chills, severe itching, a rapid pulse rate, exhaustion, anemia, weight loss and muscle weakness. It is an object of this invention to treat and to restore the skin's barrier function, prevent further loss of fluid, stabilize the body's temperature and restore the skin's chemical balance. Imbalances can put excessive pressure on the heart and kidneys, especially in older people. Because this form can be life threatening, medical care must begin immediately. People with von Zumbusch pustular psoriasis are acutely ill and often require hospitalization. Bed rest, mild sedation, bland topical therapy, rehydration and avoidance of excessive heat loss can improve the situation. Sometimes antibiotics are prescribed in case an infection is also present. However, in acute cases in which the person becomes exhausted from recurring outbreaks of pustulosis, other systemic drugs may be needed. Acitretin (Soriatane) or methotrexate is often prescribed. Cyclosporine, while FDA approved only for severe plaque psoriasis, has been used successfully in some cases of pustular psoriasis. Oral steroids are prescribed for those who do not respond to other treatments, or who have become very ill, but their use is controversial because von Zumbusch psoriasis can be triggered by the sudden withdrawal of steroids. PUVA may be used after the severe stage of pustulosis has passed.

Localized Pustules of psoriasis which are usually confined to local areas, particularly the hands and feet can be treated with either the spray on formulation or the non-spray version. One form of this psoriasis has large (up to 0.5 cm, or about the size of a pencil eraser) pustules in fleshy areas of hands and feet, such as the base of the thumb and the sides of the heels. The pustules appear in a studded pattern throughout reddened plaques of skin and then turn brown and peel. Another rare form is called acropustulosis (or acrodermatitis continua of Hallopeau). In this type, skin lesions develop on the ends of the fingers and sometimes the toes. The lesions can be painful and disabling, with nail deformities and, in severe cases, changes to the bone. Both forms of skin ailment can be treated with the formulation as the composition normalizes and stabilizes the skin cells.

Another skin ailment which can be treated with the composition is seborrheic dermatitis, which is an inflammation in areas of the body like the scalp, sides of the nose, eyebrows, eyelids, and the skin behind the ears and middle of the chest. What all these areas have in common is a high concentration of sebaceous (oil producing) glands. Other areas, such as the navel and skin folds under the arms, breasts, groin and buttocks, may also be involved. The affected skin is red and is covered by yellowish, greasy-appearing scales. Itching may or may not occur. When it does, it is usually mild. Dandruff is sometimes considered to be seborrheic dermatitis, but in fact dandruff is characterized by excessive scaling on the scalp. There is no skin inflammation. The word “seborrhea” (literally “oil flow”) describes excessive oiliness of the skin, especially on the scalp and face. There is no redness and scaling. Patients with seborrhea may later develop seborrheic dermatitis. Seborrheic dermatitis can occur at any age, but is most common in three distinct age groups infancy, when it's called “cradle cap,” middle age, and old age. Cradle cap in infancy usually clears without treatment by age eight to 12 months. This may be due to the gradual disappearance of hormones passed from the mother to the child before birth. Gentle shampooing is helpful. In some children, this condition may develop only in the diaper area where it can be confused with other forms of diaper rash. When seborrheic dermatitis develops at other ages, it may appear, disappear and then reappear. Whether treated or not, this condition comes and goes. Because this is such a common disorder, it's not surprising that some patients may have other skin or systemic diseases. There is an increased incidence in adults with conditions of the nervous system such as Parkinson's disease and in some patients recovering from stressful medical conditions such as heart attack. Those who have been confined to hospitals or nursing homes for long periods of time and those with immune system disorders, such as AIDS, appear to be more likely to develop seborrheic dermatitis. Some more intense forms of this condition can be seen in those with psoriasis. People with seborrheic dermatitis have no increased risk of other skin diseases. This condition does not progress to, or cause skin cancer, no matter how long it remains untreated. While it may subside without treatment, it usually improves temporarily with treatment. In any case, this condition tends to recur. If the rash is a cosmetic problem or if symptoms such as itching are significant, it should be treated. If the scalp is involved, frequent shampooing is usually recommended once the condition clears up. This skin disorder is treatable but may recur periodically, requiring re-treatment. One of the more effective methods of treatment is low strength hydrocortisone (cortisol) applied to the affected areas of skin. For patients who do not respond to this treatment, there are several other effective medications that a doctor can prescribe. The frequent use of nonprescription shampoos containing tar, zinc pyrithione, selenium sulfide, sulfur and/or salicylic acid may be recommended. Recent evidence suggests that this skin disorder may be intensified or perpetuated by a yeast-like organism. This organism is normally found on non-diseased skin in low numbers. With the increased scaling and retention of oil in seborrheic dermatitis, this yeast grows to very high numbers and can aggravate the inflammation of the disease. Specific prescription creams and shampoos can be helpful in controlling the condition in some people.

Embodiments of the invention further relate to a method for treating acne with a unique emu formulation. Acne usually begins when the body starts to produce the hormones called androgens. When androgen production goes into high gear about age 11 to 14 years acne also goes into high gear. Androgens cause the sebaceous gland to enlarge, and this is normal. People who develop acne have sebaceous glands that are over stimulated by androgens. Young women tend to have acne flare-ups that coincide with the hormonal changes associated with their menstrual cycle. These changes affect the sensitivity of their sebaceous glands to androgens.

In yet another embodiment, the invention relates to a method for treating microcomedo with the unique formulation. The first and smallest type of lesion is a clogged pore called a microcomedo. This tiny comedo occurs at the earliest stages when the follicle walls are just beginning to be stretched by trapped sebum. Microcomedones are so small that they cannot be seen without a microscope. If the microcomedo continues to develop and then ruptures, an inflammatory lesion will occur. If a microcomedo doesn't rupture, it can progress into either an open comedo (blackhead) or closed comedo (whitehead), both of which are noninflammatory In an open comedo, or blackhead, the contents of the follicle are firmly impacted inside.

The tightly compacted cells and sebum give the follicle its ‘black’ appearance. The dark color does not indicate the presence of dirt. Blackheads can't be washed away. They can, however, be removed with a comedo extractor by a physician. They should not be squeezed, as squeezing can irritate the skin and cause or spread inflammation. Closed comedones, or whiteheads, are skin-colored or white, and just look like small bumps on or under the skin.

Inflammatory acne lesions are the ones that have a red color, often making a ring around the pimple itself. They result when sebaceous material from the comedo gets into the surrounding tissue and causes an inflammatory reaction. Picking and squeezing acne lesions is a frequent cause of lesion rupture and inflammation, which increases the risk for scarring. For this reason, it is important to leave acne lesions alone. The invention can treat these lesions and made them vanish.

There are basically three types of inflammatory acne lesions: papules, pustules and nodules. Papules and pustules are relatively small, while nodules are larger, more severe acne lesions. Papules (typical pimples) are small, firm, reddish lesions and are sometimes considered an intermediate step between noninflammatory lesions and clearly inflammatory lesions. Papules are mildly inflamed, showing redness but no apparent pus. All can be treated with the unique formulation.

Acne is often described as one of two types: papulopustular or nodular. The most common type is papulopustular acne, which is a combination of comedones and pustules or papules. In other words, this type of acne exhibits both noninflammatory and mildly inflammatory lesions. “Acne vulgaris” is another term often used to refer to common, ordinary acne. The severity of a particular case of papulopustular acne is determined by the ratio of comedones to papules and pustules. The more papules and pustules in comparison to comedones, the more severe the acne. This formulation works well as a spray on treatment for acne and as an applied coating to stop the spread of the acne.

Embodiments of the invention further relate to a method for treating herpes, such as herpes zoster or “shingles”. This form of herpes is caused by reactivation in the adult years of the chicken pox virus that occurred during childhood (the varicalla-zoster virus). The virus can be reactivated when the body's immunity to the virus breaks down. This may happen due to normal aging, or the body's immune system may become weakened due to stress from illness, physical or emotional stress, fatigue, poor nutrition, certain medications, chemotherapy, radiation therapy, or other factors.

Once reactivated, the virus travels along nerve fibers, usually settling in fairly isolated areas of skin on one side of the body. The infected area of the body usually has severe pain, itching, redness, numbness, and the development of a rash. The rash on the skin develops into small, fluid-filled blisters called vesicles. Within a few days of their appearance on the skin, the vesicles break open and form scabs. In severe cases, the rash can leave permanent scars, long standing pain, numbness, and skin discoloration.

Transmission of a virus from patients with herpes zoster to produce chickenpox has occurred, but occurs less frequently than transmission of virus from patients with chickenpox. Localized herpes zoster requires secretion precautions to guard against spreading of infection by direct contact with secretions from vesicles and from secretion-contaminated articles. The first symptom of zoster is burning pain, tingling or extreme sensitivity in one area of the skin usually limited to one side of the body. This may be present for one to three days before a red rash appears at that site. There may also be fever or headache. The rash soon turns into groups of blisters that look a lot like chicken pox. The blisters generally last for two to three weeks. The blisters start out clear but then pus or dark blood collects in the blisters before they crust over (scab) and begin to disappear. The pain may last longer. It is unusual but possible to have pain without blisters or blisters without pain.

Other types of skin ailments can be treated with the compositions described herein, including the treatment of herpes simplex. This form of herpes is commonly referred to as cold sores or fever blisters. It is a viral infection of the skin that may occur once or return again and again. This is because when the virus is cleared from the skin by the immune system it hides in the nerves and is never completely removed from the body. Because of the presence of the linoleic acid in the emu oil, and because of the ability to spray on the formulation, the nerves of the body can be treated with the formulation to reduce the presence of herpes.

There are two kinds of herpes virus, type 1 and type 2. Type 1 virus causes 60% of the cold sores so common on the lips and face. The other 40% of cases are caused by type 2. On the genitalia these percentages are reversed, that is 40% of genital herpes cases are caused by type 1 and 60% of cases are caused by type 2. Herpes simplex is transmitted by sexual contact, kissing, or other close contact. Family members should not share towels or linen with someone who has an outbreak of herpes on the genitalia or cold sores. Herpes can spread from person to person even when an infected individual has no outbreak or symptoms. Fever, sun exposure and menstruation can act as trigger factors for the herpes, which cause the virus to travel down the nerves to the skin and cause the outbreak known as herpes or cold sores.

Still anther ailment, which has recently been in the news is cause by Anthrax. Anthrax is an acute infectious disease caused by the spore-forming bacterium Bacillus anthracis. This formulation can treat Anthrax on the skin. Anthrax most commonly occurs in wild and domestic lower vertebrates (cattle, sheep, goats, camels, antelopes, and other herbivores), but it can also occur in humans when they are exposed to infected animals or tissue from infected animals. Anthrax infection can occur in three forms: cutaneous (skin), inhalation, and gastrointestinal. B. anthracis spores can live in the soil for many years, and humans can become infected with anthrax by handling products from infected animals or by inhaling anthrax spores from contaminated animal products or from a terrorist that placed spores in a contained space, such as an airplane. This formulation is very helpful to treat anthrax infections which occur when the bacterium enters a cut or abrasion on the skin. Skin infection tends to begin as a raised itchy bump that resembles an insect bite but within 1-2 days develops into a vesicle and then a painless ulcer, usually 1-3 cm in diameter, with a characteristic black necrotic (dying) area in the center. Lymph glands in the adjacent area may swell. About 20% of untreated cases of cutaneous anthrax will result in death.

Smallpox, an acute, highly contagious viral disease that is often fatal can be contained with this spray on formulation by killing the bacteria on the skin while not killing or drying out the underlying skin. Once considered a fairly prevalent disease that developed in epidemics around the world, smallpox now appears to have been generally controlled although recent outbreaks have occurred in the United States. High fever, prostration, back and muscle pain, and sometimes vomiting can occur with the onset of this ailment. A characteristic rash develops two to five days later on the face, the palms, and the soles of the feet. During the next six to ten days the rash develops into pustular (pus-filled) pimples which can also be treated by the novel emu formulation, particularly the spray on version. In extreme cases the pustular pimples run together, which usually indicates a lethal infection of the virus. The return of fever and related symptoms initiates the second stage of disease, during which the pustules may become secondarily infected by bacteria. As recovery begins, the pustules become crusted, often leaving scars, and the fever and related symptoms subside. The composition can be used to control the blisters through the term of the disease.

One of the most common skin infections in children is impetigo and it can also be treated with this formulation. Although impetigo is most common in children, adults can also become infected. Many people think this is a problem that only concerns people who are poor or living in unsanitary conditions, but anyone can become infected with impetigo. An open wound such as an abrasion, insect bite, or minor injury can turn into impetigo. The germ that causes impetigo is Streptococcal. This bacteria of often present in sand and soil. Dirty fingers can infect broken skin through casual contact and itching. Scratching occurs with this infection and crusts that form from scratching should be removed by soaking the area with warm water and then treating with the emu formulation. Very severe cases of impetigo can cause eczema. Severe forms of impetigo can also form crusts on the skin that are dark in color and deep.

With this ailment, usually a cluster of small blisters or red bumps appears first at the site of infection. These blisters will grow and rupture within about twenty-four hours. The formulation can be used to treat these blisters.

D. Products

Embodiments of the present invention further include an elastomeric article. The elastomeric article includes an elastomeric substrate having a first side and a second side opposite the first side. The elastomeric article further includes an absorbent layer flexibly bonded to at least a first portion of the first side, wherein the absorbent layer includes an emu oil based composition, as described above.

The elastomeric article can further include an adhesive composition applied to the second portion and a third portion of the elastomeric substrate. The adhesive composition can include a pressure sensitive adhesive adapted to removably adhere to skin. The third portion generally occupies a different portion of the elastomeric substrate than the first and second portion. For example, the compositions described herein can be applied to a band-aid or other therapeutic devices. Alternatively, the second portion can surround the first portion. For example the compositions described herein can be applied to a therapeutic patch for the skin.

Embodiments of the invention can further include applying the compositions described herein to a plurality of fibers, the plurality of fibers being adapted for cleansing and treating skin by wiping the plurality of fibers over the skin.

Embodiments of the invention further include an absorbent article. The absorbent article includes a liquid permeable top sheet, wherein the liquid permeable top sheet is adapted to contact skin and is treated with an emu oil based composition, as described herein. The absorbent article further includes a liquid permeable back sheet and an absorbent core disposed between the liquid permeable top sheet and the liquid permeable back sheet.

E. Treatment of Animal Ailments

Embodiments of the invention further include a composition for treating animal ailments. The composition can be any of the compositions described herein that are adapted for topical application to animals to treat and relieve symptoms of skin ailments, such as wounds. Many animals can be treated with the composition, such as horses, dogs, farm animals and pets.

The composition can further include a lick preventor. The lick preventor can include any lick preventor known to one skilled in the art, such as aloe, tannin or bitter apple.

Embodiments of the invention further include a method for treating animal ailments. The method includes applying the emu oil based compositions described herein to an area of the animal's skin, wherein the area of the animal's skin includes the ailments.

In one embodiment, the invention includes a method to prevent licking of ailments on animals. The method generally includes preparing an emu oil based composition, as described herein, and applying the emu oil based composition to the ailment, wherein the emu oil based composition is adapted to treat the wound and prevent disturbance of the emu oil based composition by the pet.

### Hypothetical Example

An emu oil based composition will be prepared having the composition of Table 2.

Fist allantoin, methylparaben and propylparaben are dissolved in benzyl acohol. Refined sterilized emu oil, along with benzoin, and alkyl esters are added to the solution. The solution is then agitated until well blended.

TABLE 2

| | |
|---|---|
| Refined sterilized emu oil | 63.5 wt. % |
| benzyl alcohol | 20 wt. % |
| benzoin | 10 wt. % |
| allantoin | 2 wt. % |
| methylparaben | 1.25 wt. % |
| propylparaben | 0.4 wt. % |
| lineolic acid | 31 wt. % |
| alkyl ester | 3 wt. % |

The composition can then be analyzed and will likely have the properties listed in table 3.

TABLE 3

| | |
|---|---|
| fatty acid | 0.02% |
| peroxide | 0.3 units |
| water | 0.02% |
| Iodine | 72.8 mEq/100 g |
| OSI (?) | 11.95 hours @ 110° C. |
| odor | none |
| color | none |

The composition can then be sprayed on an area of a person having a bacterial infection. It is contemplated that the wound area will heal in less time than with conventional remedies and with less scarring than conventional remedies.

While the foregoing is directed to the preferred embodiments of the present invention, other and further embodiments of the invention can be devised without departing from the basic scope thereof, and the scope thereof is determined by the claims that follow.

What is claimed is:

1. A therapeutic composition comprising:
an emu oil based composition, wherein the therapeutic composition is adapted for cleansing and treating skin and wherein the emu oil based composition comprises:
a. from about 20 wt. % to about 75 wt. % of refined sterilized emu oil;
b. from about 0.01 wt. % to about 33 wt. % of one or more aromatic alcohols;
c. from about 10 wt. % to about 25 wt. % of one or more benzoic acids;
d. from about 0.2 wt. % to about 2.0 wt. % of allantoin;
e. from about 0.025 wt. % to about 1.25 wt. % of methylparaben;
f. from about 1.0 wt. % to about 13.0 wt. % of one or more alkyl esters; and
g. from about 0.01 wt. % to about 0.30 wt. % of propylparaben.

2. The therapeutic composition of claim 1, wherein the therapeutic article is adapted for cleansing and treating the skin ailments of children.

3. The therapeutic composition of claim 2, wherein the emu oil composition comprises 5 wt. % or less of the one or more aromatic alcohols.

4. The therapeutic composition of claim 1, wherein the skin ailments are selected from the group consisting of herpes, impetigo, psoriasis, seborrheic dermatitis, acne, micromedo, anthrax, diaper rash and combinations thereof.

5. The therapeutic composition of claim 1, wherein the emu oil is refined to a color of white.

6. The therapeutic composition of claim 1, wherein the emu oil is refined to a contaminant level of about 3% or less.

7. The therapeutic composition of claim 1, wherein the at least one of the one or more aromatic alcohols comprise sterilized benzyl alcohol.

8. The therapeutic composition of claim 1, wherein at least one of the one or more aromatic alcohols are independently selected from the group consisting of benzyl alcohol, propyl alcohol, methyl alcohol and combinations thereof.

9. The therapeutic composition of claim 1, wherein the one or more benzoic acids comprise benzoin.

10. The therapeutic composition of claim 1, wherein at least one of the one or more alkyl esters is branched.

11. The therapeutic composition of claim 1, wherein the refined sterilized emu oil comprises one or more fatty acids independently selected from the group consisting of monosaturated fatty acids, unsaturated fatty acids, polysaturated fatty acids, polyunsaturated fatty acids, essential fatty acids and combinations thereof.

12. The therapeutic composition of claim 11, wherein the essential fatty acids are independently selected from the group consisting of myristic acid, palmitic acid, palmitoleic acid, margaric acid, margaric oleic acid, oleic acid, linoleic acid, linolenic acid, arachidic acid, eicosinoac acid and combinations thereof.

13. The therapeutic composition of claim 1, wherein the emu oil comprises iodine.

14. The therapeutic composition of claim 1, wherein the composition is adapted to strengthen and rebuild skin.

15. The therapeutic composition of claim 1, wherein the composition is stable when exposed to air.

16. The therapeutic composition of claim 1, wherein the composition comprises from about 60 wt. % to about 70 wt. % of the refined sterilized emu oil.

17. The therapeutic composition of claim 16, wherein the composition comprises from about 15 wt. % to about 20 wt. % of the one or more aromatic alcohols.

18. The therapeutic composition of claim 16, wherein the composition comprises from about 1 wt. % to about 1.25 wt. % of the methylparaben.

19. The therapeutic composition of claim 16, wherein the composition comprises from about 0.25 wt. % to about 0.3 wt. % of the propylparaben.

20. The therapeutic composition of claim 16, wherein the composition comprises from about 1.5 wt. % to about 2.0 wt. % of the allantoin.

21. The therapeutic composition of claim 16, wherein the composition comprises from about 2.5 wt. % to about 3 wt. % of the one or more alkyl esters.

22. The therapeutic composition of claim 1, wherein the therapeutic composition is applied to a plurality of fibers and adapted for cleansing and treating skin by wiping the therapeutic article over the skin.

23. The therapeutic composition of claim 1, wherein the therapeutic composition is adapted for cleansing and treating skin by spraying the therapeutic composition on the skin.

24. The therapeutic composition of claim 1, wherein the therapeutic composition is adapted for cleansing and treating skin by rolling the therapeutic composition on the skin.

25. The therapeutic composition of claim 1, wherein the therapeutic composition is adapted to inhibit bacterial growth on skin.

26. A composition comprising:
an aqeous emu oil based composition, wherein the aqueous emu oil based composition is adapted for cleansing and treating skin and wherein the aqueous emu oil based composition comprises;
a. from about 20 wt. % to about 75 wt. % of refined sterilized emu oil;
b. from about 0.01 wt. % to about 33 wt. % of one or more aromatic alcohols;
c. from about 10 wt. % to about 25 wt. % of one or more benzoic acids;
d. from about 0.2 wt. % to about 2.0 wt. % of allantoin;
e. from about 0.025 wt. % to about 1.25 wt. % of methylparaben;
f. from about 1.0 wt. % to about 13.0 wt. % of one or more alkyl esters; and
g. from about 0.01 wt. % to about 0.30 wt. % of propylparaben.

27. An absorbent article comprising:
a. a liquid permeable top sheet, wherein the liquid permeable top sheet is adapted to contact skin and is treated with an emu oil based composition, wherein the emu oil based composition comprises;
i. from about 20 wt. % to about 75 wt. % of refined sterilized emu oil;
ii. from about 0.01 wt. % to about 33 wt. % of one or more aromatic alcohols;
iii. from about 10 wt. % to about 25 wt. % of one or more benzoic acids;
iv. from about 0.2 wt. % to about 2.0 wt. % of allantoin;
v. from about 0.025 wt. % to about 1.25 wt. % of methylparaben;
vi. from about 1.0 wt. % to about 13.0 wt. % of one or more alkyl esters; and
vii. from about 0.01 wt. % to about 0.30 wt. % of propylparaben;
b. a liquid permeable back sheet; and
c. an absorbent core disposed between the liquid permeable top sheet and the liquid permeable back sheet.

28. A method for preventing baby rashes and skin ailments comprising:
a. preparing an emu oil based composition comprising:
i. from about 20 wt. % to about 75 wt. % of refined sterilized emu oil;
ii. from about 0.01 wt. % to about 33 wt. % of one or more aromatic alcohols;
iii. from about 10 wt. % to about 25 wt. % of one or more benzoic acids;
iv. from about 0.2 wt. % to about 2.0 wt. % of allantoin;
v. from about 0.025 wt. % to about 1.25 wt. % of methylparaben;
vi. from about 1.0 wt. % to about 13.0 wt. % of one or more alkyl esters; and
vii. from about 0.01 wt. % to about 0.30 wt. % of propylparaben; and
b. applying the emu oil based composition to the an ailments selected from the group consisting of baby rashes and skin ailments.
